(19) United States
(12) Patent Application Publication
(10) Pub. No.: US 2026/0249052 A1
(43) Pub. Date: Aug. 27, 2026

(54) INTRAVASCULAR ACCESS DEVICE WITH ADAPTIVE NAVIGATION

(71) Applicant: Covidien LP, Mansfield, MA (US)

(72) Inventors: Danyong ZENG, Aliso Viejo, CA (US); Maria D. SANSON, Mission Viejo, CA (US); Tasha C. ABOUFADEL, Oceanside, CA (US); Tejashri KUMAR, Fountain Valley, CA (US); Omarinda S. NICHOLS, Lakewood, CA (US)

(21) Appl. No.: 19/550,026

(22) Filed: Feb. 25, 2026

Related U.S. Application Data
(60) Provisional application No. 63/764,085, filed on Feb. 27, 2025.

(51) Int. Cl. A61M 25/06; A61M 25/00

(52) U.S. Cl. CPC A61M 25/0662; A61M 25/0045; A61M 25/0054; A61M 25/0068; A61M 2210/127

(57) ABSTRACT

Treatment devices and associated methods and systems are disclosed herein. According to some embodiments, an access device comprises an elongated member. The access device may comprise a plurality of cuts disposed between a proximal end and a distal end of the elongated member along a helical axis extending around the elongated member. The access device may comprise a plurality of connectors each separating adjacent cuts along the helical axis, wherein a number of cuts per rotation varies along a longitudinal dimension of the elongated member. The access device may comprise a first region with a first rotational cut pattern and a second region with a second rotational cut pattern offset in a direction opposite the first rotational cut pattern. In some embodiments, a configuration of the cuts along a length of the elongated member facilitates navigation through complex anatomical structures.

### CROSS-REFERENCE TO RELATED APPLICATIONS

This application claims the benefit of U.S. Provisional Patent Application Serial No. 63/764,085, filed February 27, 2025, the entire content of which is incorporated herein by reference.

### TECHNICAL FIELD

The present technology relates to systems and methods for removing obstructions from body lumens. Some embodiments of the present technology relate to laser-cut hypotubes and associated components.

### BACKGROUND

Many medical procedures use access device(s) to guide a medical device to a target location (such as clotting material or another obstruction) within a body lumen, vessel, or other organ. When a target location is in distal, tortuous anatomy such of that of the intracranial vasculature, it becomes challenging to deliver a treatment device to the target location. Medical devices can be introduced using access devices (e.g. catheters); however, use of these devices presents challenges. An access device must be sufficiently flexible so as to navigate the tortuous anatomy, but sufficiently stiff so as to exhibit the desired properties of pushability and kink resistance. Laser-cut hypotubes including cut patterns varying in pitch along the length of the hypotube have been developed to overcome this issue as they exhibit improved kink resistance during delivery and aspiration, but this feature alone is insufficient to adequately access complex anatomical environments, particularly in neurovascular applications. In addition, certain intricate anatomical structures such as the aortic bend, middle cerebral artery (MCA), common carotid artery (CCA), and/or internal carotid artery (ICA) can cause access devices to twist and lock in place, preventing further advancement. Accordingly, there remains a need for improved devices and methods for navigating such complex anatomy.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic diagram illustrating an access system according to some embodiments.

FIG. 2 is a block diagram illustrating an embodiment of an access device.

FIG. 3A is a side perspective view of a portion of an access device according to some embodiments.

FIG. 3B is an enlarged detailed view of a portion of the access device shown in FIG. 3A.

FIG. 3C is a planar representation of connector configurations along the access device shown in FIGS. 3A and 3B.

FIGS. 4–6 are graphs showing planar representations of various regions of access devices comprising variable connector configurations according to some embodiments.

FIG. 7 is a graph showing the variation in pitch along the length of an access device according to some embodiments.

FIG. 8 is a graph showing the variation in cut pattern along the length of an access device according to some embodiments.

FIG. 9 is a graph showing the variation in connector pattern along the length of an access device according to some embodiments.

FIG. 10 is a graph showing the variation in stiffness by region along the length of an access device according to some embodiments.

### DETAILED DESCRIPTION

The present disclosure relates generally to systems and methods for navigating devices to target locations (e.g. treatment sites) in bodily lumens such as blood vessels, and more particularly to flexible access devices configured to retain structural integrity while navigating complex anatomy. Traditional guidewires and access catheters often lack the versatility necessary to navigate certain anatomical structures, which can lead to challenges in accessing treatment sites such as those located within the distal neurovasculature. For example, traditional laser-cut catheters typically comprise a cut pattern oriented in one rotational direction (e.g. oriented clockwise about a longitudinal axis of the device). This configuration can result in the catheter locking as a result of twisting due to navigation through complex structures, thereby preventing further advancement of the access device.

The access devices described herein address these shortcomings through a variable cut pattern configured to promote both flexibility and pushability as the device is advanced through complex anatomy. It may be advantageous to provide an access device having a greater stiffness at a proximal region than at a distal region. In some embodiments, the access device may comprise a guidewire and/or a catheter. In some embodiments, the access device has a cut pattern varying in pitch along its length. The cut pattern may comprise a larger pitch size in a proximal region of the access device and a smaller pitch size in a distal region of the access device. In some embodiments, the pitch may increase more rapidly in some regions and more gradually in other regions. It may be advantageous to provide an access device having a faster torque response at a proximal region than at a distal region. In some embodiments, the access device has a variable number of cuts per rotation (e.g. the number of cuts disposed along a circumference of the device) along its length. For example, the access device may comprise a greater number of cuts per rotation at a distal region than at a proximal region. It may be advantageous to provide regions of greater stiffness along the length of the access device to balance pushability and torque response. In some embodiments, the cut pattern includes connectors which can be aligned at variable intervals along the length of the access device. The connectors may be aligned with greater frequency at a distal region of the device and with less frequency at a proximal region of the device. It may be advantageous to provide access devices resistant to a twisting effect, which may cause the device to lock during introduction, preventing further advancement to distal anatomical structures. In some embodiments, the access device comprises at least one region with a cut pattern oriented in a first direction and at least one region with a cut pattern oriented in a second direction. The first direction may be a clockwise direction, and the second direction may be a counterclockwise direction. These features can result in increased flexibility at a distal region of the delivery device, thereby facilitating navigation through tortuous anatomy while retaining structural integrity via a stiffer proximal region.

Various other features and aspects of example access systems and devices are described in more detail below. The present technology provides systems, devices, and methods for treating various medical conditions, including accessing a target site within a bodily lumen. Although many of the embodiments are described below with respect to devices, systems, and methods for accessing the cerebral or intracranial vasculature, other applications and other embodiments in addition to those described herein are within the scope of the technology. For example, the systems and methods of the present technology may be used to access body lumens other than blood vessels (e.g., the digestive tract, etc.) and/or may be used to access blood vessels outside of the brain (e.g., pulmonary, abdominal, cervical, or thoracic blood vessels, or peripheral blood vessels including those within the legs or arms, etc.). In addition, the systems and methods of the present technology may be used to remove luminal obstructions (e.g., clots, plaque, resected tissue, foreign material, etc.) in conjunction with methods such as aspiration and/or mechanical engagement. Additionally or alternatively, the systems and devices described herein can be used for fluid delivery to a target site within a bodily lumen (e.g., delivery of medicament, saline, contrast media, or other suitable fluid to an intravascular treatment site within a cerebral vessel or other desired treatment site).

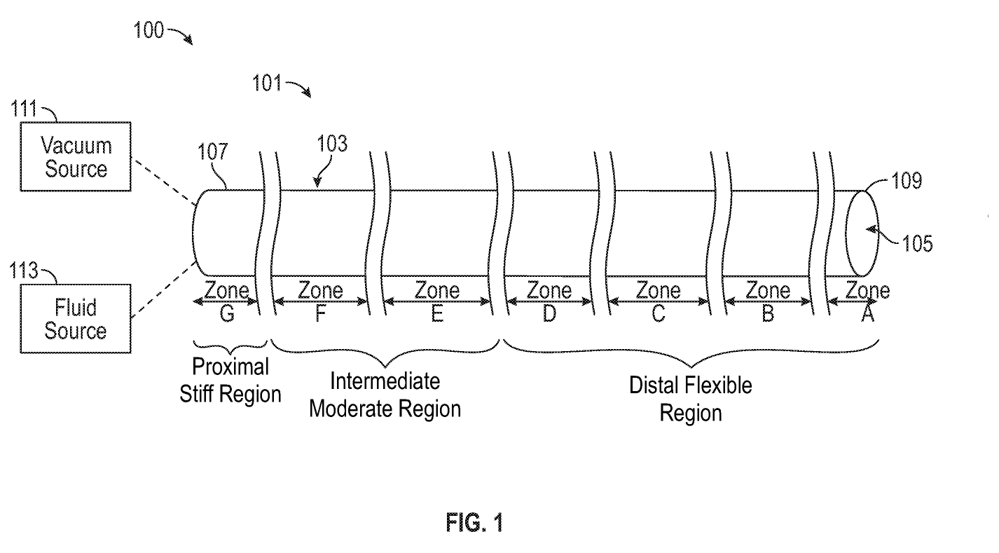

Referring to FIG. 1, a schematic side view of an exemplary system 100 is shown that may be used for minimally invasive medical procedures. In some implementations, the system 100 includes an access device 101 having a plurality of Zones (labeled Zone A through Zone G) arranged along a longitudinal axis between a distal end 109 and a proximal end 107. The access device 101 may include an elongated tubular member 103 defining a lumen 105 that, in some implementations, comprises a laser-cut hypotube having various patterns and configurations along its length to provide desired mechanical properties.

The plurality of Zones may be arranged into three main regions: a distal flexible region, an intermediate moderate region, and a proximal stiff region. In some implementations, the distal flexible region may be configured to navigate through complex vasculature such as neurovascular anatomy. The intermediate moderate region may provide a transition in mechanical properties, while the proximal stiff region may be configured to navigate through larger vessels such as the aortic arch.

For general intravascular applications, the elongated member 103 can have an outer diameter ranging from about 3 French (Fr) to about 24 Fr, and an inner diameter ranging from about 0.013 inches to about 0.200 inches, depending on the target vessel size and intended use. In implementations configured specifically for neurovascular applications, the elongated member 103 can have a smaller profile with an outer diameter ranging from about 3 Fr to about 8 Fr at its distal end, corresponding to approximately 1.0 mm to 2.7 mm, and an inner diameter ranging from about 0.020 inches to about 0.088 inches to enable navigation through the tortuous vessels of the neurovasculature while maintaining sufficient lumen size for clot aspiration. Additionally or alternatively, the elongated member 103 can have a tapered configuration with a larger diameter at its proximal end transitioning to a smaller diameter at its distal end to balance trackability with aspiration efficiency. In some embodiments, the outer diameter and/or inner diameter remain substantially constant along the length of the elongated member 103.

In some embodiments, the system 100 includes one or more elements operably coupled to the access device 101. These elements may be positioned extracorporeally and/or intracorporeally. Additional elements that could be used in conjunction with the present delivery devices include a vacuum source 111 configured to supply negative pressure to a target location and/or a fluid source 113 configured to deliver fluid (e.g. saline, contrast, therapeutic agents, or combinations thereof) to a target location. The components of the system 100 can be arranged in various configurations while maintaining similar functionality. In some embodiments, a vacuum source and fluid source can be integrated into a single unit. In some implementations, the access device 101 includes a hub assembly at the proximal end 107 that may interface with auxiliary equipment such as the vacuum source 111 and/or the fluid source 113. The hub assembly may include one or more ports for connecting to such equipment.

In some embodiments, the elongated member 103 is a laser-cut hypotube, with the cut pattern optionally varying along the length of the elongated member 103 to modify flexibility characteristics. The elongated member 103 may include an inner liner formed from fluoropolymers such as polytetrafluoroethylene (PTFE) or fluorinated ethylene propylene (FEP) to provide a lubricious inner surface and can optionally include hydrophilic or hydrophobic coatings on exterior surfaces to enhance trackability through vasculature.

The elongated member 103 may include various structural features along its length. As described in more detail below, these features can include cut patterns which define include connectors arranged in different patterns within each Zone. For example, connectors in certain Zones may be oriented in a clockwise direction while connectors in other Zones may be oriented in a counterclockwise direction to facilitate navigation through specific anatomical features. Additionally or alternatively, connectors heights can vary across or within Zones. In various implementations, the elongated member 103 may include a varying pitch size in its laser-cut pattern, such as increasing from the distal end 109 toward the proximal end 107. By varying these and other features of the elongated member 103, the desired combination of mechanical features can be provided to enable navigation of tortuous anatomy such as the cerebral neurovasculature.

The system 100 may optionally include a guidewire that extends through the lumen 105. In some embodiments, the guidewire can be used to navigate the access device 101 through tortuous vasculature to reach a target location. The guidewire can be removably disposed within the lumen 105 such that it can be withdrawn or advanced relative to the access device 101 during a procedure. In some embodiments, the system 100 does not include a guidewire and the access device 101 may be navigated to the target location independently.

The access device 101 may also include an inner liner extending through the lumen 105 and/or an outer jacket surrounding the elongated member 103. In some implementations, the outer jacket may comprise different materials and/or different durometers along the length of the access device to further enhance the desired mechanical properties of each region. In some implementations, the outer jacket may comprise the same material and/or durometer along the length of the access device.

The various Zones and regions of system 100 work in concert to provide a device capable of navigating through complex anatomical pathways while maintaining pushability, torque response, and kink resistance. The combination of varying laser-cut patterns, connector orientations, and material properties enables the access device to be used in procedures with or without a guidewire, providing flexibility in surgical approaches.

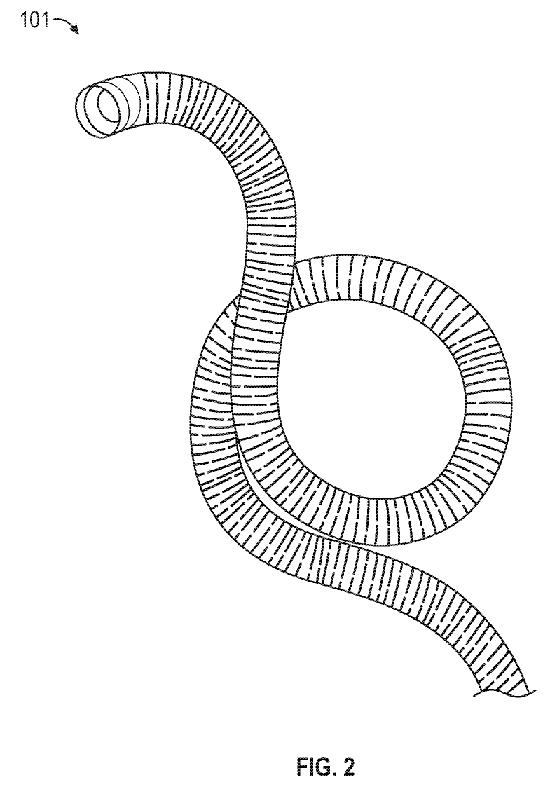

FIG. 2 shows an access device 101 (e.g., a hypotube or catheter) according to some embodiments of the present technology in a looped configuration. The access device 101 may therefore be capable of navigation through anatomical structures such as the aortic bend, the MCA, the CCA, and/or the ICA. As described in greater detail herein, various embodiments of access devices may include a plurality of regions comprising different cut patterns to facilitate the arrangement of access devices in such configurations.

As noted above, optimizing flexibility and pushability is a challenge currently faced by conventional access systems, particularly when accessing complex and/or distal anatomy such as that of the neurovasculature. The access devices of the present disclosure address such challenges, for example, by providing variable cut patterns configured to optimize material properties along various regions of an access device 101 and/or facilitate the navigation of various regions of an access device 101 through complex anatomical structures. In some embodiments, the cut pattern is formed through laser-cutting, although other methods may be used to generate the structures disclosed herein.

As noted above, the elongated member 103 may comprise a plurality of regions or Zones longitudinally spaced from one other, wherein the regions comprise different material properties. Such variations may allow the access device 101 to be customizable for various applications pertaining to accessing a bodily lumen, particularly through complex anatomical structures such as those present within the neurovasculature.

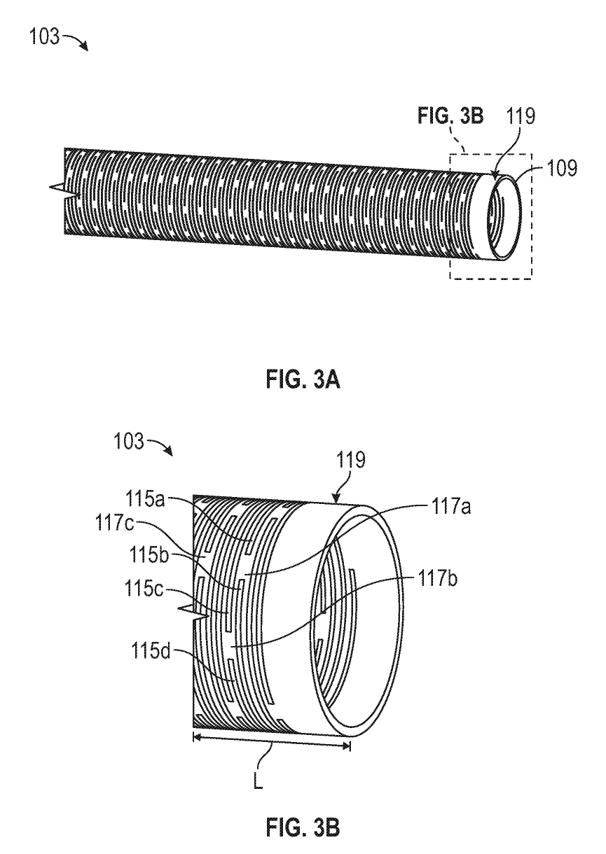

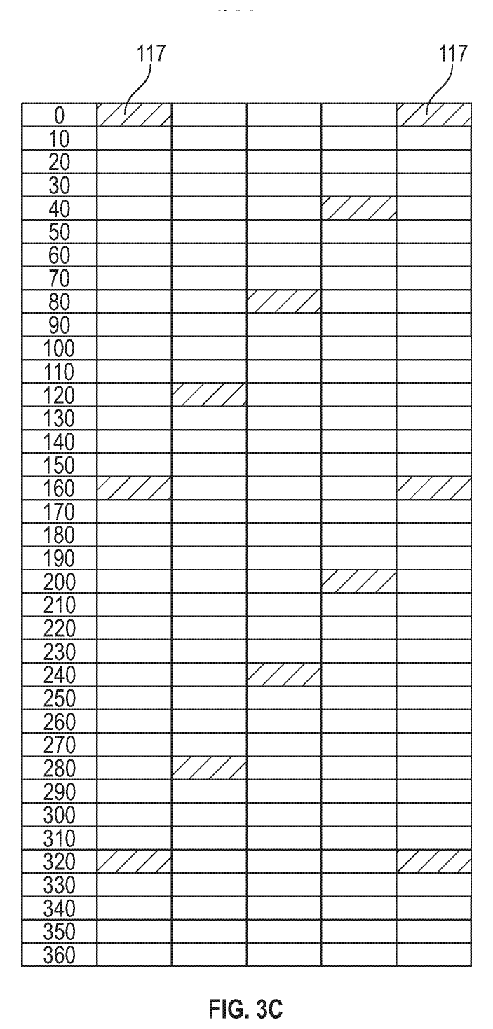

FIG. 3A is a side perspective view of a portion of the elongated member 103 including Zone A (defined by uncut portion 119 at the distal end 109) and a portion of Zone B (which includes a plurality of flexibility-enhancing cuts 115 extending helically around the elongated member 103).

Referring to FIG. 3B, a detailed view of a distal portion of the elongated member 103 is shown with a length extending along a longitudinal axis L. In some implementations, the elongated member 103 includes a plurality of cuts 115 (individually labeled as 115a–115d) formed within the body of the elongated member 103. The cuts 115 may be separated by connectors 117 (individually labeled 117a–117c), which represent uncut portions of the elongated member 103.

In some implementations, the cuts 115a–115d are arranged in a helical pattern around the circumference of the elongated member 103. The pitch of this pattern may be defined as the number of cuts per unit length along the longitudinal axis L. For example, in some implementations, the pitch may range from about 2.25 cuts per rotation at a distal portion to about 3.2 cuts per rotation at a proximal portion of the elongated member 103.

The connectors 117 may be characterized by a connector height, which represents the length of each connector measured along the helical axis on which the cuts 115 are oriented. In some implementations, the connector height may vary along the length of the elongated member 103 to provide different mechanical properties in different Zones, for instance increasing from a distal region to a proximal region, further optimizing the pushability of the access device 101.

Additionally or alternatively, the connectors 117 may be arranged in specific patterns defined by their circumferential alignment and rotational positioning. For example, a first connector 117a may be circumferentially aligned with a third connector 117c, separated by a connector alignment spacing measured in number of rotations around the longitudinal axis L (here the connectors 117a and 117c are aligned with 3 intervening rotations, and so the connector pattern repeats every 4 rotations in this example). In some implementations, this alignment spacing may range from about 3–4 rotations in a distal portion to about 10–12 rotations in a proximal portion of the elongated member 103.

The connectors may also follow a connector bridge pattern defined by the rotational offset between connectors in adjacent rotations of the helical axis. This offset may create either a clockwise or counterclockwise progression of connectors along the length of the elongated member 103. In some implementations, the connector bridge pattern may change from a clockwise orientation to a counterclockwise orientation in different Zones of the elongated member 103 to facilitate navigation through specific anatomical features.

FIG. 3C shows a graphical representation of the connector layout pattern that along Zone B of the elongate member 103. The vertical axis reflects the angular position around the circumference at which a given connector 117 is positioned, and each column represents a single rotation of the helical cut pattern.

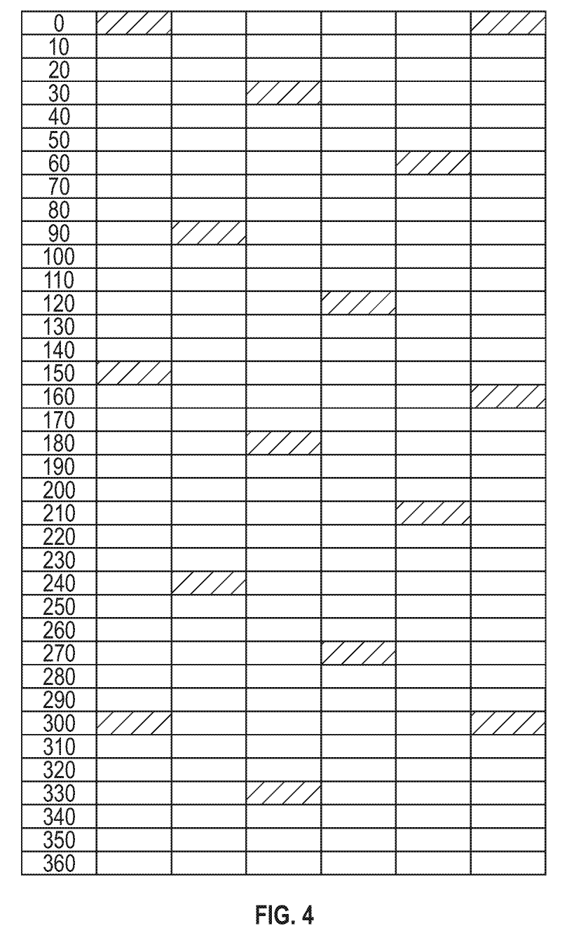

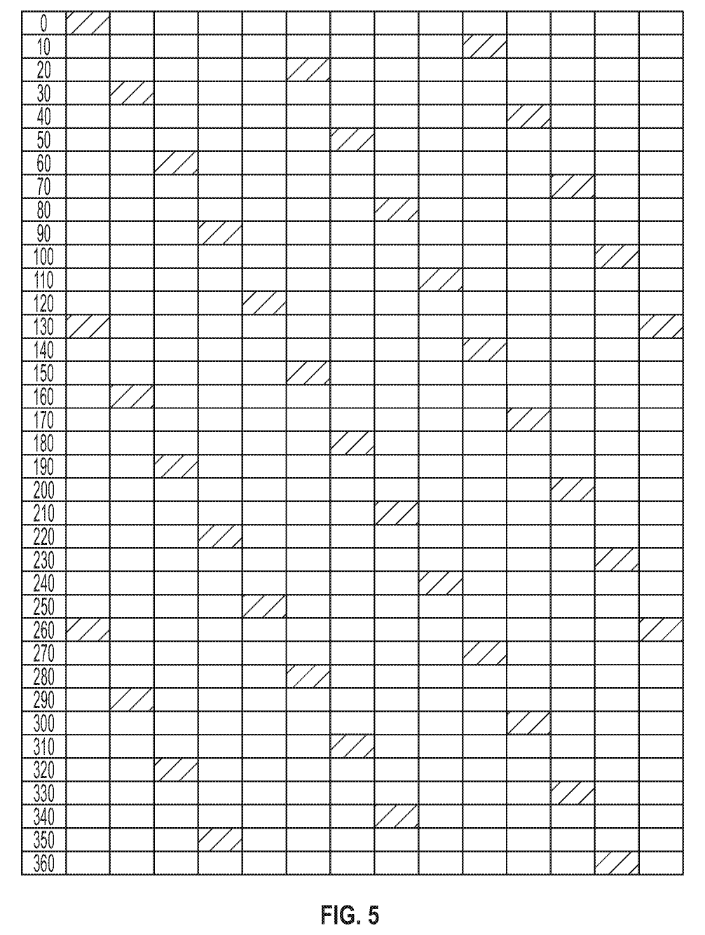

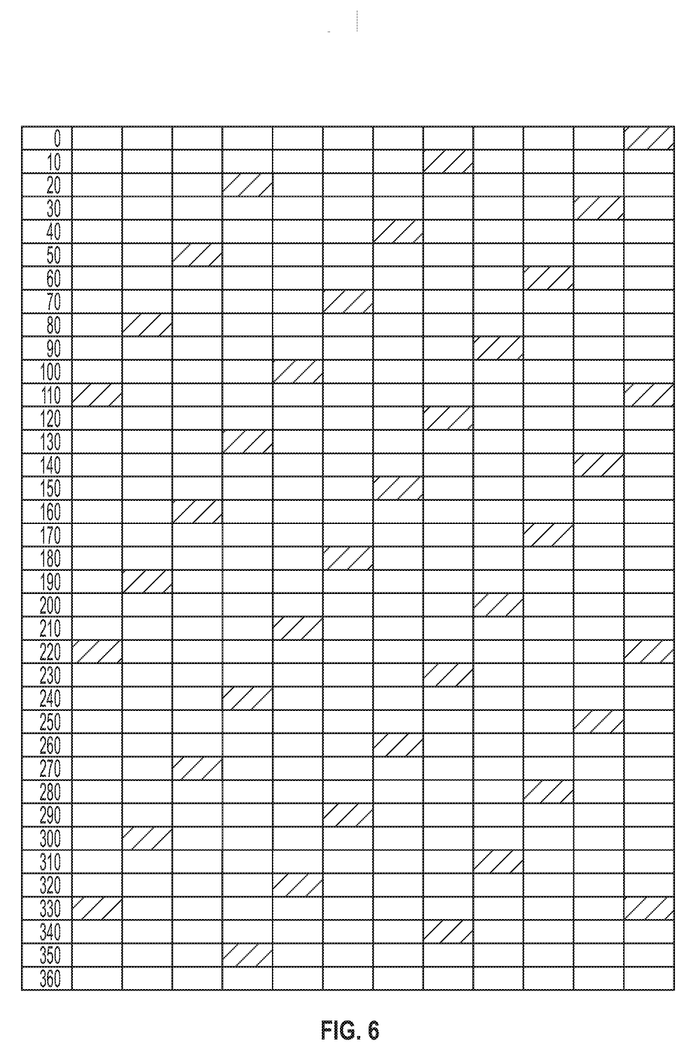

FIGS. 4, 5, and 6 show graphical representations of the connector layout patterns for Zone E (FIG. 4), Zone F (FIG. 5), and Zone G (FIG. 6), respectively. As noted previously, Zone G can be a Zone of maximum stiffness (e.g., comprising the proximal stiff region), while Zones E and F can have an intermediate stiffness (with Zone E being more flexible than Zone F).

The specific combinations of pitch, connector height, connector alignment spacing, and connector bridge patterns may be varied along the length L to achieve desired mechanical properties such as flexibility, pushability, and torque response in different Zones of the elongated member 103. In some embodiments, the elongated member 103 comprises various regions or Zones (e.g., Zones A–G depicted in FIG. 1) with different cut patterns configured to facilitate navigation through various anatomy. The cut patterns may be configured to provide a flexibility that gradually decreases from a distal region to a proximal region of the elongated member 103.

Some regions of an elongated member 103 may comprise connectors 117 oriented in a first rotational direction (e.g., clockwise), while other regions or Zones of the elongated member 103 can have connectors 117 oriented in a second, opposite rotational direction (e.g., counterclockwise). Compare, for example, the connector bridge pattern in FIG. 3C (Zone B) with the connector bridge pattern in FIG. 5 (Zone F). In the bridge pattern in FIG. 3C, for a given connector 117, a nearest connector 117 on the adjacent rotation is offset in the upward direction, while the inverse is true in the connector bridge pattern of FIG. 5.

In some embodiments, an elongated member 103 comprises at least one region with a connector bridge pattern oriented in a clockwise direction and at least one region with a connector bridge pattern oriented in a counterclockwise direction. Providing regions with different rotational connector bridge patterns may help to alleviate any twisting effect during navigation of the access device 101 to a target location. In some embodiments, a region of the elongated member 103 comprising a connector bridge pattern orientation opposite that of adjacent regions of the elongated member 103 may be configured to be positioned within the aortic bend.

Another characteristic of the connector pattern is the length or number of rotations between circumferential alignment of connectors 117. The connectors 117 may be substantially circumferentially aligned with each other along the longitudinal axis L of an access device 101 at variable numbers of rotations. In some embodiments, the variation in the configuration of the connectors 117 contributes to an access device 101 that gradually decreases in flexibility from a distal region to a proximal region. Closer connection in a flexible distal region and farther connection in a stiff proximal region may assist the device in smooth navigation through the anatomy, resulting in consistent performance. In some embodiments, the number of rotations per connector alignment increases from about 3 or 4 at a distal region to about 10 or 12 at a proximal region. In some embodiments, the distal region comprises a number of rotations per connector alignment from about 1 to about 3, from about 3 to about 4, from about 4 to about 6, from about 6 to about 8, from about 8 to about 10, or from about 1 to about 10. In some embodiments, the distal region comprises a number of rotations per connector alignment less than 1 or greater than 10. In some embodiments, the proximal region comprises a number of rotations per connector alignment from about 6 to about 8, from about 8 to about 10, from about 10 to about 12, from about 12 to about 14, from about 14 to about 16, or from about 6 to about 16. In some embodiments, the proximal region comprises a number of rotations per connector alignment less than 6 or greater than 16. In some embodiments, the connectors 117 are oriented with a20degree phase difference at each rotation (e.g. a first rotation comprises connectors 117 disposed at 0, 130, and 260 degrees and a second rotation comprises connectors 117 disposed at 30, 160, and 290 degrees as seen in FIG. 5). In some embodiments, the connectors 117 are oriented with a 30 degree phase difference at each rotation (e.g. a first rotation comprises connectors 117 disposed at 110, 220, and 330 degrees and a second rotation comprises connectors 117 disposed at 80, 190, and 300 degrees as seen in FIG. 6). In some embodiments, the connectors 117 are oriented with a 40 degree phase difference at each rotation (e.g. a first rotation comprises connectors 117 disposed at 0, 160, and 320 degrees and a second rotation comprises connectors 117 disposed at 120 and 180 degrees as seen in FIG. 3C). In some embodiments, the connectors 117 are oriented with a 60 degree phase difference at each rotation (e.g. a first rotation comprises connectors 117 disposed at 0, 150, and 300 degrees and a second rotation comprises connectors 117 disposed at 90 and 240 degrees as seen in FIG. 4).

With reference back to FIG. 1, in some embodiments the access device 103 has a plurality of Zones A–G which generally increase in stiffness in the proximal direction. For instance, in some embodiments the distal flexible region including Zones A–D is most flexible, the proximal stiff region including Zone G is least flexible, and the intermediate moderate region including Zone E and Zone F has an intermediate stiffness. Various aspects of these various Zones and regions are described in more detail below.

Beginning with the distal flexible region, which includes Zones A–D, this region comprises the distal end of the elongated member 103. The first region may comprise the greatest flexibility of the access device 101, which facilitates navigation through the tortuous anatomy of the neurovasculature. In some implementations, the first region may comprise about 2.35 cuts per rotation.

Zone A comprises a tip portion 119 disposed at the distalmost end of the elongated tubular member 103. The tip portion 119 may comprise no cuts 115, which allows the tip portion 119 to provide structural integrity to the distal terminus of the elongated member 103. In some embodiments, the tip portion 119 is formed integrally with the elongated member 103 and in some embodiments the tip portion 119 is a separate element. The tip portion 119 may be radiopaque, allowing for more precise placement within a target location. In some embodiments, the tip portion 119 comprises a length from about 0.2 mm to about 0.5 mm.

Zone B may be configured to navigate through the MCA. In some embodiments, Zone B comprises a length from about 50 mm to about 100 mm. In some embodiments, the cut pattern along Zone B increases in pitch from about 0.0035 inches at a distal portion to about 0.004 inches at a proximal portion. In some embodiments, Zone B comprises a connector layout corresponding to FIG. 3C.

Zone C can comprise a low pitch size that gradually increases in a proximal direction. In some embodiments, Zone C comprises a pitch size greater than a pitch size of the Zone B. In some embodiments, the pitch size increases more rapidly in Zone C than in the Zone B. Zone C may provide a balance of flexibility and pushability to facilitate navigation of the access device 101 through moderate bends. For example, in some embodiments, Zone C is configured to navigate through the MCA and/or ICA. In some embodiments, Zone C comprises a length from about 50 mm to about 80 mm. In some embodiments, Zone C increases in pitch from about 0.004 in at a distal portion to about 0.0045 in at a proximal portion.

Next is Zone D, which may comprise a low pitch size that gradually increases in a proximal direction, as well as a connector height that is higher than in Zone C. In some embodiments, Zone D comprises a pitch size greater than a pitch size of Zone C. In some embodiments, the pitch size increases more rapidly in Zone D than in Zone C. Zone D may be configured to navigate through the ICA and/or CCA. In some embodiments, Zone D comprises a length from about 80 mm to about 120 mm. In some embodiments, Zone D increases in pitch from about 0.0045 inches at a distal portion to about 0.0055 inches at a proximal portion.

Zone E may be disposed proximally of Zone D, and forms part of the intermediate moderate stiffness region of the access device 101. Zone E may increase in pitch size in a proximal direction, and may increase in pitch size more rapidly than in Zones B–D, providing the access device 101 with additional support and flexibility. In some embodiments, Zone E is moderately flexible. In some embodiments, the second region is configured to navigate through the CCA. In some embodiments, the second region comprises a length from about 100 mm to about 150 mm. In some embodiments, the second region increases in pitch from about 0.0055 in at a distal portion to about 0.0085 at a proximal portion. Zone E may comprise a connector layout corresponding to FIG. 4. In some embodiments, Zone E comprises a greater number of cuts per rotation than in any of Zones B–D. The number of cuts per rotation in Zone E may be about 2.65.

Zone F may be disposed proximally of Zone E. As shown in FIG. 1, together Zone E and Zone F can define the intermediate moderate stiffness region. Zone F may increase in pitch size in a proximal direction, and may increase in pitch size more rapidly than Zone E, providing the access device 101 with additional support and flexibility. In some embodiments, Zone F is moderately stiff. In some embodiments, Zone F is configured to navigate from the CCA to the aortic arch. In some embodiments, Zone F comprises a length from about 80 mm to about 120 mm. In some embodiments, Zone F increases in pitch from about 0.0085 inches at a distal portion to about 0.00115 inches at a proximal portion. Zone F may comprise a connector layout corresponding to FIG. 5. In some embodiments, Zone F comprises connectors 117 that are offset from each other in a rotational direction opposite of that of adjacent regions (e.g., the connectors 117 of the third region are offset from each other in a counterclockwise direction while the connectors 117 of adjacent regions are offset from each other in a clockwise direction).

Zone G may be disposed proximally of Zone F, and in some embodiments Zone G defines the proximal end of the elongated member 103. Zone G may increase in pitch size in a proximal direction, and may increase in pitch size more rapidly than Zone F, providing the access device 101 with additional support. In some embodiments, Zone G is relatively stiff. In some embodiments, Zone G is configured to navigate from the femoral vein to the aortic arch. In some embodiments, Zone G comprises a length from about 200 mm to about 1200 mm. In some embodiments, Zone G increases in pitch from about 0.00115 in at a distal portion to about 0.002 at a proximal portion. Zone G may comprise a connector layout corresponding to FIG. 6.

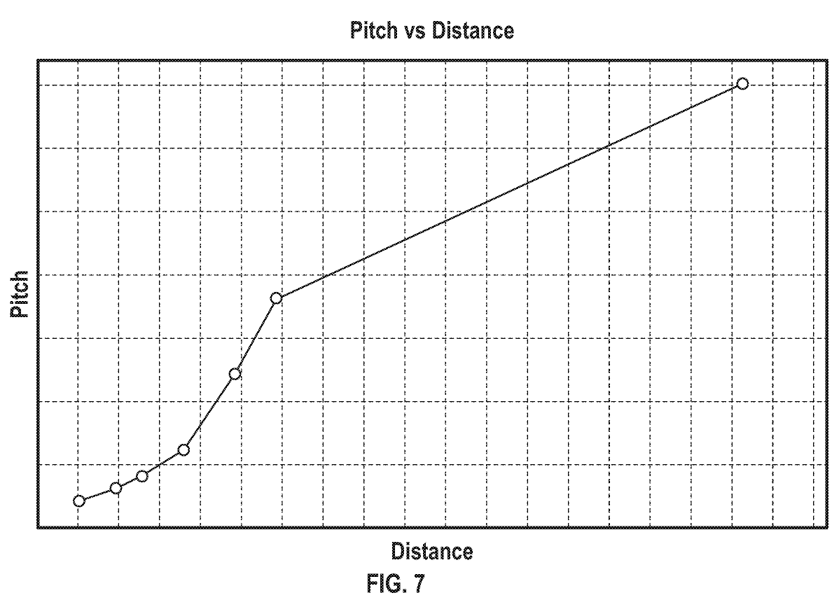

FIG. 7 is a graph showing the variation in pitch of the cut pattern on an access device 101 in relation to distance from the distal end of the access device 101 according to some embodiments. It may be advantageous to provide an elongated member 103 with a pitch that varies along the longitudinal axis L. As used herein, the term “pitch” refers to the longitudinal separation between the cuts 115 in the sidewall of the elongated member 103. A smaller pitch size may correspond to a greater flexibility while a larger pitch size may correspond to a greater stiffness of a region of an elongated member 103. According to some embodiments as shown in FIG. 7, the pitch gradually increases from a distal region to a proximal region of the elongated member 103, creating a stiffness gradient in which the distal region is more flexible and the proximal region is stiffer. This stiffness gradient may facilitate navigation through anatomical pathways by allowing the access device 101 to adapt to variable levels of resistance presented by the anatomy. The change in pitch may be uniform, or it may vary along the length of the elongated member 103. In some embodiments, the pitch changes gradually in a region configured to be placed distally of the MCA. In some embodiments, the pitch changes more rapidly in a region configured to be positioned within the MCA, ICA, and/or CCA to provide flexibility for navigating the complex anatomy. In some embodiments, the pitch changes gradually in a region configured to be placed proximally of the aortic bend to provide structural support in a location comprising relatively straight anatomy.

In some embodiments, the pitch size increases from about 0.0035 inches at a distal region of the elongated member 103 to about 0.02 inches at a proximal region of the elongated member 103. In some embodiments, the distal region comprises a pitch size from about 0.001 inches to about 0.002 inches, from about 0.002 inches to about 0.0035 inches, from about 0.0035 inches to about 0.005 inches, from about 0.005 inches to about 0.010 inches, or from about 0.001 inches to about 0.010 inches. In some embodiments, the distal region comprises a pitch size less than 0.001 inches or greater than 0.010 inches. In some embodiments, the proximal region comprises a pitch size from about 0.010 inches to about 0.015 inches, from about 0.015 inches to about 0.020 inches, from about 0.020 inches to about 0.025 inches, from about 0.025 inches to about 0.030 inches, or from about 0.010 inches to about 0.030 inches. In some embodiments, the proximal region comprises a pitch size less than 0.010 inches or greater than 0.030 inches.

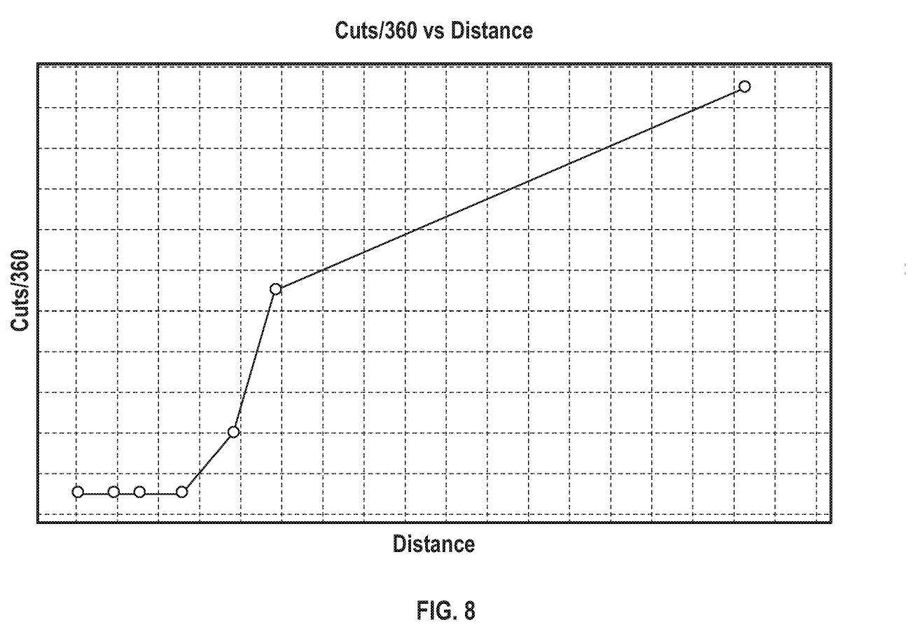

FIG. 8 is a graph showing the variation in the number of cuts per rotation on an access device 101 in relation to distance from the distal end of the access device 101 according to some embodiments. It may be advantageous to provide an elongated member 103 with a number of cuts per rotation that varies along the longitudinal axis L. As used herein, a “rotation” refers to a circumferential dimension of an elongated member 103 (i.e., a region having 2.5 cuts per rotation refers to a region in which an average of 2.5 cuts span a distance equivalent to the circumference of the elongated member 103). In other words, a region having a greater number of cuts per rotation may indicate that the individual cuts within that region are shorter in length than the cuts in another region. A greater number of cuts per rotation may correspond to a faster torque response while a lesser number of cuts per rotation may correspond to a slower torque response. In some embodiments, the number of cuts per rotation gradually increases from a distal region of the elongated member 103 to a proximal region of the elongated member 103. Accordingly, the proximal region of the elongated member 103 may have a faster torque response than the distal region. This may help limit torque response delay and provide smoother and more controlled movement, further improving navigation as the access device 101 is advanced through the anatomy. This feature may be particularly beneficial in anatomy comprising one or more loops, such as the aortic bend. In some embodiments, the number of cuts per rotation increases from about 2.25 at a distal region to about 3.25 at a proximal region. In some embodiments, the distal region comprises a number of cuts per rotation from about 1.5 to about 2.0, from about 2.0 to about 2.25, from about 2.25 to about 2.4, from about 2.4 to about 2.8, or from about 1.5 to about 2.8. In some embodiments, the distal region comprises a number of cuts per rotation less than 1.5 or greater than 2.8. In some embodiments, the proximal region comprises a number of cuts per rotation from about 2.8 to about 3.0, from about 3.0 to about 3.25, from about 3.25 to about 3.4, from about 3.4 to about 3.8, from about 3.8 to about 4.0, or from about 2.8 to about 4.0. In some embodiments, the proximal region comprises a number of cuts per rotation less than 2.8 or greater than 4.0.

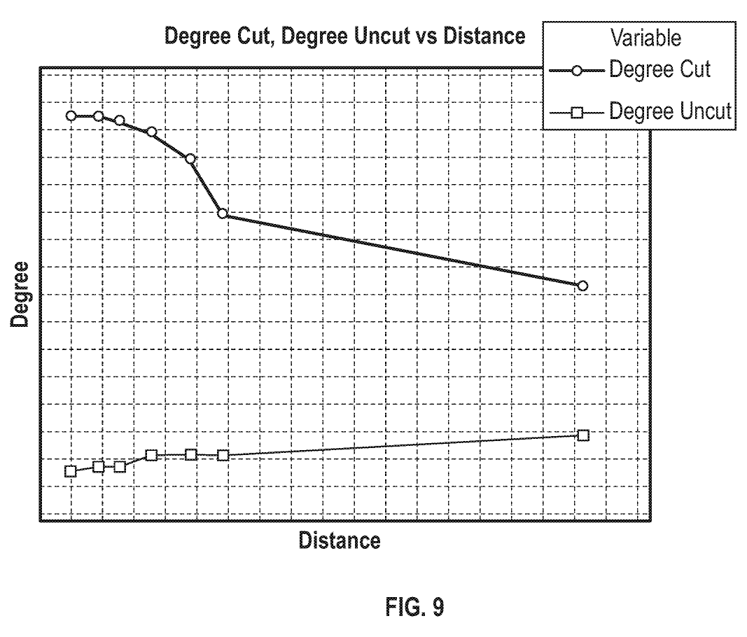

FIG. 9 is a graph showing the variation in the cut length on an access device 101 in relation to distance from the distal end of the access device 101 according to some embodiments. As used herein, “degree cut” refers to the circumferential length of a cut 115 in degrees and “degree uncut” refers to the circumferential length of a connector 117 in degrees. For example, an access device 101 may comprise a cut 115 spanning about 145 degrees at a distal end and cuts 115 spanning smaller circumferential lengths at a proximal region, resulting in a variation in the number of rotations per connector alignment along the length of the access device 101. It may be advantageous to provide an elongated member 103 with a number of rotations per connector alignment that varies along the longitudinal axis L. As used herein, the “number of rotations per connector alignment” refers to the number of cuts 115 present between two circumferentially aligned connectors 117 along the longitudinal axis L of the access device 101. For example, FIG. 3B shows a first connector 117a and a third connector 117c being aligned along the longitudinal axis L with three cuts per rotation. (i.e. connectors 117a and 117b are separated by three cuts 115). A greater number of rotations per connector alignment may correspond to greater stiffness while a lesser number of rotations per connector alignment may correspond to greater flexibility. In some embodiments, the number of rotations per connector alignment gradually increases from a distal region to a proximal region of the elongated member 103. This may help to balance pushability with torque response.

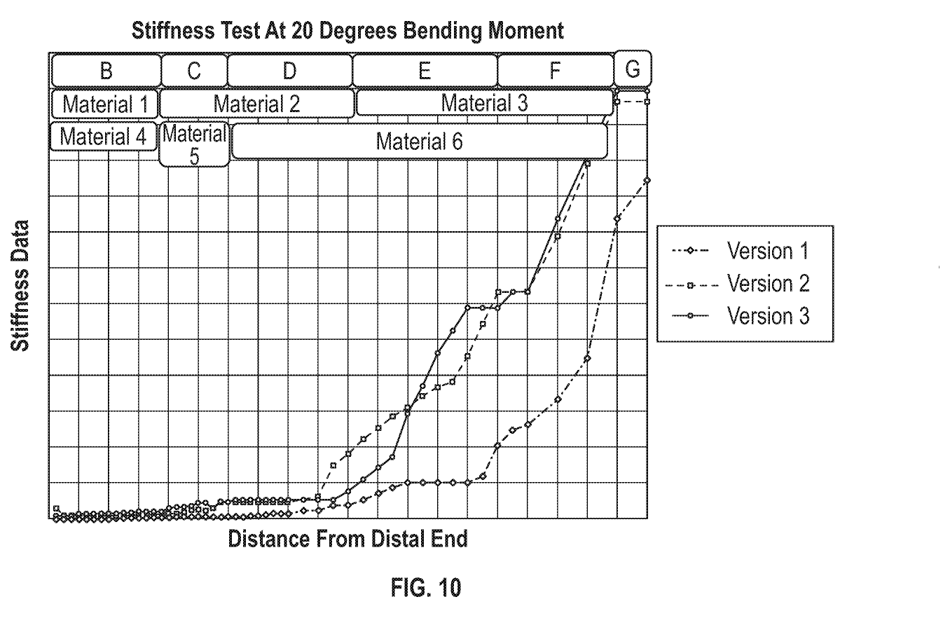

FIG. 10 shows the variation in stiffness of an access device 101 in relation to the distance from the distal end for three different versions of an access device 101 as described herein. In addition to the cut patterns described above, the access device 101 can have various jacket configurations. In the illustrated examples, the utilized jacket materials include one or more of a Material 1, Material 2, Material 3, Material 4, Material 5, or Material 6. The jacket may be configured as a single layer of materials or multiple layers of materials. By arranging these different materials in different segments of the jacket, the overall stiffness of the access device 101 can be modulated to achieve the desired characteristics. In some embodiments, relatively soft and flexible materials are disposed at a distal portion of the access device 101 while relatively hard and stiff materials are disposed at a proximal portion of the access device 101. A gradient of gradually decreasing softness and flexibility may be provided between the distal portion and proximal portion of the access device 101. In some embodiments, Material 1 is used over Zone B, Material 2 is used over Zones C and D, and Material 3 is used over Zones E and F. In some embodiments, Material 1 is NEUSOFT62, Material 2 is PEBAX 35, and Material 3 is PEBAX 55, although other configurations are possible. In some embodiments, Material 4 is used over Zone B, Material 5 is used over Zone C, and Material 6 is used over Zones D, E, and F. In some embodiments, Material 4 is NEUSOFT 62, Material 5 is PEBAX 35, and Material 6 is PEBAX 55, although other configurations are possible. While these examples illustrate discrete transitions between the hardness and/or stiffness of jacket materials that align with Zone transitions, in some cases the jacket transitions need not align precisely with Zone transitions (as defined by cut patterns described previously). Additionally or alternatively, in some cases the jacket material can be configured to have gradual transitions in hardness or stiffness, with the jacket generally being harder and more stiff in the proximal portions of the access device and softer and more flexible in the distal portions of the access device.

As noted previously, the overall stiffness of the access device generally increases with distance from the distal end, due both to variations in cut patterns and jacket material (optionally the liner material can also be varied in a manner similar to that of the jacket material). As illustrated in FIG. 9, Zones B, C, and D (which together can form the distal flexible region) exhibit a low stiffness. Zone E comprises a stiffness greater than a stiffness of any on Zones B, C, or D. Zone F comprises a stiffness greater than a stiffness of Zone E. Together, Zone E and Zone F can form the intermediate region of the device which has a moderate stiffness (e.g., more stiff than the distal flexible region but less stiff than the proximal stiff region). Finally, Zone G is the stiffest region of the device and can comprise the proximal stiff region. In some embodiments, the gradual increase in stiffness from the distal to proximal regions of the access device 101 results from the variable cut and connector configurations described previously herein. In some embodiment, the arrangement of jacket material (and/or other such coatings, liners, sheaths, layers, etc.), such as that shown in FIG. 10, may contribute to the variable flexibility and pushability of the access device 101.

While specific embodiments are described herein, it should be appreciated that the disclosed features may be combined in any suitable configuration. For example, while the referenced embodiments describe the various regions in a specific longitudinal arrangement, other arrangements are possible. The regions disclosed herein may be arranged in any suitable order to provide an access device 101 that is customizable for navigation through any specific anatomy. For example, in applications outside of the neurovasculature, it may be advantageous to provide the regions in a configuration different from those described herein. It should also be noted that the graphs presented herein are not necessarily to scale and merely represent example implementations of the present technology. The dimensions discussed herein are provided as examples and should not be construed as limiting the scope of the present technology.

The access devices disclosed herein may be used in conjunction with various methods for navigating through a patient’s anatomy. In some embodiments, the access device is used to access a bodily lumen during a medical procedure. In some embodiments, the medical procedure comprises a thrombectomy, an ablation, an occlusion, a medical device implantation, a medical device removal, an obstruction removal, a colonoscopy, or an intravascular imaging procedure.

In some embodiments, a method comprises navigating an access device 101 through the distal neurovasculature. The access device 101 may be introduced through the femoral vein and navigated to the aortic arch. In some embodiments, Zone G is configured to provide relative stiffness when positioned in this part of the anatomy. In some embodiments, Zone F comprises a pitch oriented in a rotational direction opposite that of the pitch of adjacent regions such that Zone F is configured to be positioned within the loop of the aortic arch (see FIG. 1) without impeding further navigation of the access device 101 through the vasculature. The access device 101 may be advanced from the aortic arch to the CCA. In some embodiments, the decrease in pitch size of Zone E facilitates navigation through this increasingly complex anatomy. As the access device advances further to the ICA and the MCA, the further decrease in pitch of the flexible first region disposed at the distal end of the access device 101 facilitates smooth, controlled navigation. In some embodiments, Zone D is configured to navigate through the CCA and/or ICA. In some embodiments, Zone C is configured to navigate through the ICA and/or MCA. In some embodiments, Zone B is configured to navigate through the MCA. In some embodiments, Zone A comprises a radiopaque marker to allow for improved visualization of the access device 101 within the anatomy. After the access device 101 is sufficiently advanced, a medical procedure may be performed (e.g., aspiration, delivery of fluids, delivery of a medical device, delivery of energy to a treatment site (e.g., heat, electrical, optical, or mechanical energy, such as for purposes of cavitation, ablation, remodeling etc.), or other suitable medical procedure).

### Examples

The subject technology is illustrated, for example, according to various aspects described below, including with reference to FIGS. 1–10. Various examples of aspects of the subject technology are described as numbered examples (1, 2, 3, etc.) for convenience. These are provided as examples and do not limit the subject technology.

Example 1. An access device, comprising:

an elongated member having a proximal end, a distal end, and a lumen extending therebetween;

a plurality of cuts disposed between the proximal end and the distal end, the cuts arranged along a helical axis extending around the elongated member; and

a plurality of connectors each separating adjacent cuts along the helical axis, wherein:

a number of cuts per rotation varies along a longitudinal dimension of the elongated member,

a first region of the elongated member has a first rotational connector pattern in which, for each connector, the nearest connector in the distal direction is offset in a first rotational direction,

a second region of the elongated member has a second rotational connector pattern in which, for each connector, the nearest connector in the distal direction is offset in a second rotational direction, wherein the second rotational direction is opposite to the first rotational direction, and

wherein a configuration of the cuts along a length of the elongated member facilitates navigation through complex anatomical structures.

Example 2. The access device of Example 1, wherein a proximal region of the elongated member comprises a stiffness greater than a stiffness at a distal region of the elongated member.

Example 3. The access device of Example 1 or Example 2, wherein a height of the connectors decreases from the proximal end to the distal end of the elongated member.

Example 4. The access device of any one of Examples 1–3, wherein the connectors are arranged in a connector pattern such that individual connectors are circumferentially aligned with one another every n rotations, and wherein the number of rotations n separating circumferentially aligned connectors decreases from a proximal end to the distal end of the elongated member.

Example 5. The access device of Example 4, wherein the number of rotations separating circumferentially aligned connectors decreases from at least 10 rotations to 4 or fewer rotations along the length of the elongated member.

Example 6. The access device of any one of Examples 1–5, wherein the number of cuts per rotation is greater in a proximal region of the elongated member than in a distal region of the elongated member.

Example 7. The access device of any one of Examples 1–6, wherein the cuts comprise a pattern that increases in pitch from the proximal end to the distal end of the elongated member.

Example 8. The access device of any one of Examples 1–7, wherein the elongated member is a laser-cut hypotube.

Example 9. An access device, comprising:

a tubular body having a proximal end and a distal end, wherein the tubular body includes a plurality of zones arranged along a longitudinal axis between the proximal end and the distal end, the plurality of zones comprising:

a distal zone having a first connector pattern with connectors oriented in a first rotational direction;

a proximal zone having a second connector pattern with connectors oriented in the first rotational direction; and

an intermediate zone between the distal zone and the proximal zone, the intermediate zone having a third bridge connector pattern with connectors oriented in a second rotational direction opposite the first rotational direction, wherein the intermediate zone is configured to navigate through an aortic arch.

Example 10. The access device of Example 9, wherein the distal zone comprises connectors aligned every three to four rotations.

Example 11. The access device of Example 9 or Example 10, wherein the proximal zone comprises connectors aligned every ten to twelve rotations.

Example 12. The access device of any one of Examples 9–11, wherein the distal zone connector pattern repeats every five rotations.

Example 13. The access device of any one of Examples 9–12, wherein the intermediate zone connector pattern repeats every thirteen rotations.

Example 14. The access device of any one of Examples 9–13, wherein in the distal zone:

the connectors of a first rotation are positioned at 0 degrees, 160 degrees, and 320 degrees; and

the connectors of a second rotation are positioned at 120 degrees and 280 degrees.

Example 15. The access device of any one of Examples 9–14, wherein in the intermediate zone:

the connectors of a first rotation are positioned at 0 degrees, 130 degrees, and 260 degrees; and

the connectors of a second rotation are positioned at 30 degrees, 160 degrees, and 290 degrees.

Example 16. The access device of any one of Examples 9–15, wherein the tubular body comprises a laser-cut pattern having a pitch size that increases from the distal end toward the proximal end.

Example 17. The access device of Example 16, wherein the pitch size increases from approximately 0.0035 inches at the distal end to approximately 0.02 inches at the proximal end.

Example 18. The access device of any one of Examples 9–17, wherein the number of cuts per rotation increases from the distal end toward the proximal end.

Example 19. The access device of Example 18, wherein the number of cuts per rotation increases from approximately fewer than 2.5 cuts per rotation at the proximal end to more than 3.0 cuts per rotation at the distal end.

Example 20. The access device of any one of Examples 9–19, further comprising:

a liner disposed within the tubular body; and

a jacket disposed around the tubular body.

Example 21. The access device of Example 20, wherein:

the liner comprises a lubricious polymer; and

the jacket comprises segments of different durometer materials arranged along the longitudinal axis.

Example 22. The access device of any one of Examples 9–21, wherein a connector height gradually increases from the distal end toward the proximal end within each zone.

Example 23. A method of navigating a medical device to a treatment site, comprising:

introducing an elongated member into a patient, the elongated member comprising:

a tubular body having a proximal end and a distal end, wherein the tubular body includes a plurality of zones arranged along a longitudinal axis between the proximal end and the distal end, the plurality of zones comprising:

a distal zone having a first connector pattern with connectors oriented in a first rotational direction;

a proximal zone having a second connector pattern with connectors oriented in the first rotational direction; and

an intermediate zone between the distal zone and the proximal zone, the intermediate zone having a third connector pattern with connectors oriented in a second rotational direction opposite the first rotational direction; and

navigating the elongated member to a complex anatomical structure, wherein a configuration of cuts along a length of the elongated member facilitates the navigation of the elongated member through the complex anatomical structure.

Example 24. The method of Example 23, wherein a proximal region of the elongated member comprises a stiffness greater than a stiffness at a distal region of the elongated member.

Example 25. The method of Example 23 or Example 24, wherein the complex anatomical structure comprises an aortic bend.

Example 26. The method of any one of Examples 23–25, wherein navigating the elongated member to a complex anatomical structure comprises navigating the elongated member through a loop of at least 360 degrees.

### Conclusion

Although many of the embodiments are described above with respect to systems, devices, and methods for treating vessel occlusions in the brain, the technology is applicable to other applications and/or other approaches, such as vessel occlusions elsewhere in the body and/or obstructions in other bodily lumens. As noted herein, in some implementations the treatment devices and systems disclosed herein can be used in conjunction with fluid delivery and/or aspiration systems. This can include, for instance, delivery of fluid containing medicament (e.g., any substance used for medical treatment, diagnosis, disease prevention, and/or health promotion). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1–10.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An access device, comprising:
an elongated member having a proximal end, a distal end, and a lumen extending therebetween;
a plurality of cuts disposed between the proximal end and the distal end, the cuts arranged along a helical axis extending around the elongated member; and
a plurality of connectors each separating adjacent cuts along the helical axis, wherein:
a number of cuts per rotation varies along a longitudinal dimension of the elongated member,
a first region of the elongated member has a first rotational connector pattern in which, for each connector, the nearest connector in the distal direction is offset in a first rotational direction,
a second region of the elongated member has a second rotational connector pattern in which, for each connector, the nearest connector in the distal direction is offset in a second rotational direction, wherein the second rotational direction is opposite to the first rotational direction, and
wherein a configuration of the cuts along a length of the elongated member facilitates navigation through complex anatomical structures.

2. The access device of claim 1, wherein a proximal region of the elongated member comprises a stiffness greater than a stiffness at a distal region of the elongated member.

3. The access device of claim 1, wherein a height of the connectors decreases from the proximal end to the distal end of the elongated member.

4. The access device of claim 1, wherein the connectors are arranged in a connector pattern such that individual connectors are circumferentially aligned with one another every n rotations, and wherein the number of rotations n separating circumferentially aligned connectors decreases from a proximal end to the distal end of the elongated member.

5. The access device of claim 4, wherein the number of rotations separating circumferentially aligned connectors decreases from at least 10 rotations to 4 or fewer rotations along the length of the elongated member.

6. The access device of claim 1, wherein the number of cuts per rotation is greater in a proximal region of the elongated member than in a distal region of the elongated member.

7. The access device of claim 1, wherein the cuts comprise a pattern that increases in pitch from the proximal end to the distal end of the elongated member.

8. The access device of claim 1, wherein the elongated member is a laser-cut hypotube.

9. An access device, comprising:
a tubular body having a proximal end and a distal end, wherein the tubular body includes a plurality of zones arranged along a longitudinal axis between the proximal end and the distal end, the plurality of zones comprising:
a distal zone having a first connector pattern with connectors oriented in a first rotational direction;
a proximal zone having a second connector pattern with connectors oriented in the first rotational direction; and
an intermediate zone between the distal zone and the proximal zone, the intermediate zone having a third bridge connector pattern with connectors oriented in a second rotational direction opposite the first rotational direction, wherein the intermediate zone is configured to navigate through an aortic arch.

10. The access device of claim 9, wherein the distal zone comprises connectors aligned every three to four rotations.

11. The access device of claim 9, wherein the proximal zone comprises connectors aligned every ten to twelve rotations.

12. The access device of claim 9, wherein the distal zone connector pattern repeats every five rotations.

13. The access device of claim 9, wherein the intermediate zone connector pattern repeats every thirteen rotations.

14. The access device of claim 9, wherein the tubular body comprises a laser-cut pattern having a pitch size that increases from the distal end toward the proximal end.

15. The access device of claim 9, wherein the number of cuts per rotation increases from the distal end toward the proximal end.

16. The access device of claim 9, further comprising:
a liner disposed within the tubular body; and
a jacket disposed around the tubular body.

17. A method of navigating a medical device to a treatment site, comprising:
introducing an elongated member into a patient, the elongated member comprising:
a tubular body having a proximal end and a distal end, wherein the tubular body includes a plurality of zones arranged along a longitudinal axis between the proximal end and the distal end, the plurality of zones comprising:
a distal zone having a first connector pattern with connectors oriented in a first rotational direction;
a proximal zone having a second connector pattern with connectors oriented in the first rotational direction; and
an intermediate zone between the distal zone and the proximal zone, the intermediate zone having a third connector pattern with connectors oriented in a second rotational direction opposite the first rotational direction; and
navigating the elongated member to a complex anatomical structure, wherein a configuration of cuts along a length of the elongated member facilitates the navigation of the elongated member through the complex anatomical structure.

18. The method of claim 17, wherein a proximal region of the elongated member comprises a stiffness greater than a stiffness at a distal region of the elongated member.

19. The method of claim 17, wherein the complex anatomical structure comprises an aortic bend.

20. The method of claim 17, wherein navigating the elongated member to a complex anatomical structure comprises navigating the elongated member through a loop of at least 360 degrees.
